(19) United States
(12) Patent Application Publication
(10) Pub. No.: US 2025/0353988 A1
(43) Pub. Date: Nov. 20, 2025

(54) DEPOLYMERIZATION OF LIGNIN USING A SUPPORTED METAL CATALYST

(71) Applicants: THE REGENTS OF THE UNIVERSITY OF CALIFORNIA, Oakland, CA (US); NATIONAL TECHNOLOGY & ENGINEERING SOLUTIONS OF SANDIA, LLC, Albuquerque, NM (US)

(72) Inventors: Hemant CHOUDHARY, Emeryville, CA (US); Lailitendu DAS, Emeryville, CA (US); Blake A. SIMMONS, San Francisco, CA (US); John M. GLADDEN, Alameda, CA (US); Seema SINGH, Hayward, CA (US)

(21) Appl. No.: 19/287,580

(22) Filed: Jul. 31, 2025

Related U.S. Application Data
(63) Continuation of application No. PCT/US2024/013389, filed on Jan. 29, 2024.
(60) Provisional application No. 63/483,503, filed on Feb. 6, 2023.

(51) Int. Cl. C08J 11/16; C07C 37/00

(52) U.S. Cl. CPC C08J 11/16; C07C 37/004; C08J 2397/02

(57) ABSTRACT

The present invention provides for a method for depolymerizing a lignin, said method comprising: (a) providing a metal catalyst, and (b) contacting a lignin to the metal catalyst, such that the metal catalyst depolymerizes at least a portion of the lignin into one or more lignin monomers.

### CROSS-REFERENCE TO RELATED APPLICATIONS

This application claims priority as a continuation application to PCT International Patent Application No. PCT/US2024/013389, filed Jan. 29, 2024, which claims priority to U.S. Provisional Patent Application Ser. No. 63/483,503, filed Feb. 6, 2023, both of which are hereby incorporated by reference.

### STATEMENT OF GOVERNMENTAL SUPPORT

The invention was made with government support under Contract No. DE-AC02-05CH11231 awarded by the U.S. Department of Energy. The government has certain rights in the invention.

### FIELD OF THE INVENTION

The present invention is in the field of depolymerizing lignin.

### BACKGROUND OF THE INVENTION

The rapid socio-economic development and industrialization resulted in the overutilization of the limited fossil fuel resources causing global energy crisis and thereby necessitating the transition to sustainable renewable sources (1). Among these, only lignocellulosic biomass has been identified as a sustainable feedstock for the production of renewable carbon-based fuels and chemicals owing to its composition and annual production rate (2). Lignocellulose e.g., agricultural residues, forestry and domestic wastes, and municipal solid wastes, is a complicated composite of tangled cellulose and hemicellulose enwrapped in lignin and held together by strong covalent and hydrogen bonds limiting its facile utilization (3). Several developments have been made to utilize the cellulosic (and hemicellulosic) components of the lignocellulose, however, lignin, a sustainable source of aromatics, is largely utilized as a means of generating heat and power in a biorefinery through combustion (4). To attain environmental, social, and economic sustainability, all lignocellulosic components must be utilized to their full potential, especially lignin (4, 6). Depolymerization of lignin has been widely studied for its potential to produce biofuels and bioproducts (7). Typically, lignin depolymerization results in a pool of aromatic and non-aromatic products and the development of strategic approaches has been successful in narrowing down potential outputs (8). Among the studied lignin depolymerization routes, namely oxidation, hydrolysis, photo-chemical transformations, catalytic hydrogenolysis has been identified as the most promising route due to high atom-economy (9). These depolymerization routes have focused on conversion of either holocellulosic components or the native-lignin first in the whole lignocellulose biomass. Traditional delignification approaches involving acids or bases offers maximum utilization of holocellulosic components but renders “difficult to process” or technical lignin due to the formation of stable C—C bond between phenylpropanoids via carbocation intermediates (10, 11). To address this issue, “lignin-first” approaches were developed that prevent condensation of phenylpropanoids by either catalysis or masking of labile functional groups (12). Nevertheless, compromised monomeric sugar yields along with wide array of product profile still needs to be taken care of during the lignin-first approach (8). Pretreatment of lignocellulosic biomass with ionic liquids (ILs, organic salts with a melting point below 100° C.) (13) has been identified for enhancing monomeric sugar yields and minimum structural changes of lignin (14). Among several available ILs, bioderived and biocompatible cholinium-based ILs are of general interest to biorefinery, providing an opportunity for an integrated fractionation, saccharification, and fermentation of lignocellulose (termed as “one-pot” conversion) while reducing the energy costs by 40% and leaving behind lignin for further upgradation into fuels and chemicals (15, 16, 17). Herein, we aim to develop a heterogeneous catalytic hydrogenolytic process for lignin residue obtained from the one-pot conversion of poplar biomass using cholinium lysinate ([Ch][Lys]) ionic liquid. It is important to highlight that a limited number of studies exists with a focus on depolymerizing IL-based biorefinery lignin (18, 19).

A number of lignin hydrogenolysis studies have been reported on various lignin sources, for example, hydrogenolysis have been performed over solid catalysts such as carbon, silica, zeolites, metal oxides, carbides supported transition metals namely, Ni, Cu, Fe, Pt, Pd, Rh, and Ru (8, 9, 20, 21, 22, 23). Most of these catalysts were not recycled/recyclable as the formation of char, or metal and water impurities, or condensation reactions contributes to surface saturation and deactivation. To improve the process economics, catalysts recycling is a must and development of robust catalytic processes with a) high selectivity, b) minimum char formation, c) high conversion, d) reusable and robust catalysts are still needed. Based on these lignin hydrogenolysis reports, hydrogenation of C—O and C—C bonds over transition metal catalysts was favored in the presence of a support with Bronsted acid sites (8). In another instance of C—O bond cleavage, it was suggested that the interaction of substrate with Bronsted acid sites enhance the conversion of lignin reducing undesired side-reactions, while the presence of Lewis acids stabilizes the transition state through chemisorption (24, 25, 26). In this regard metal phosphates, especially zirconium phosphate (ZrP) has attracted attention as robust solid acid catalyst (27) and have been employed for various transformations including dehydration of alcohols (28), hydrogenation (29), and hydrodeoxygenation (30, 31) reactions.

### SUMMARY OF THE INVENTION

The present invention provides for a method for depolymerizing a lignin, said method comprising: (a) providing a metal catalyst, and (b) contacting a lignin to the metal catalyst, such that the metal catalyst depolymerizes at least a portion of the lignin into one or more lignin monomers.

In some embodiments, the metal catalyst is a metal-nanoparticle (NP) catalyst. In some embodiments, the metal catalyst is supported on a solid support. In some embodiments, the NP is a multimetallic NP, such as a bimetallic NP or trimetallic NP. In some embodiments, the metal catalyst is Ni, Cu, Cr, Co, Fe, Ru, Rh, Pd, Pt, Au, Re, and/or Ir, or a mixture thereof. In some embodiments, the metal catalyst is palladium.

In some embodiments, the metal catalyst is supported on or bound to a support, such as a solid support. In some embodiments, the support is an acid support or a basic support. In some embodiments, the solid support comprises zirconium phosphate. In some embodiments, the metal catalyst is solid acid support comprising zirconium phosphate.

In some embodiments, the one or monomers are a phenol or guaiacol, or a derivative thereof, or a compound disclosed in FIGS. 1-12 (such as FIGS. 1, 2, 3, 5, 6, 11, and/or 12). In some embodiments, the one or lignin monomers comprise a

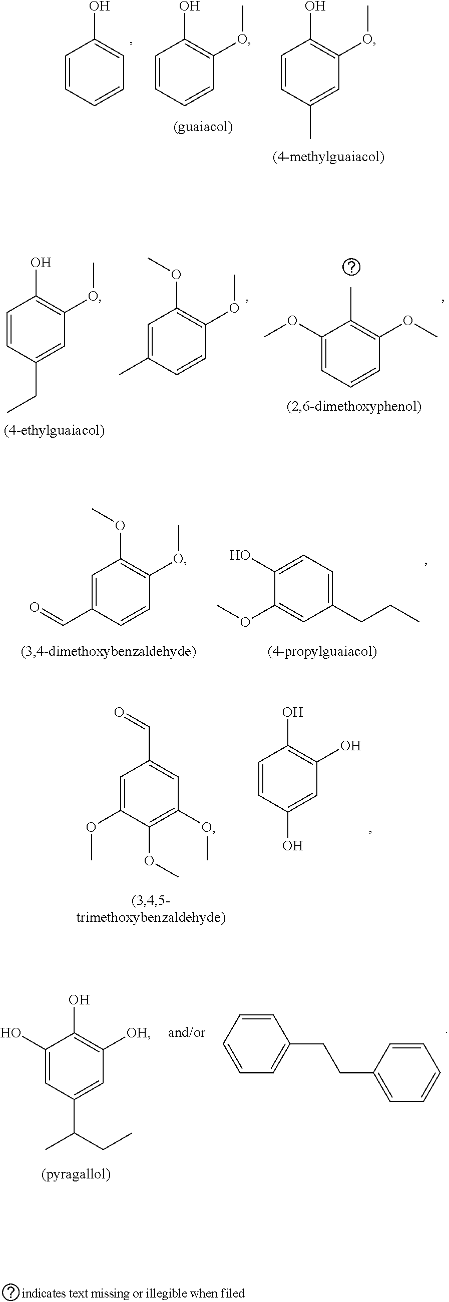

In some embodiments, the method comprises solubilizing, or deconstructing, or pretreating a biomass to obtain or release lignin from the biomass, prior to the (a) providing and (b) contacting steps. In some embodiments, the solubilizing, or deconstructing, or pretreating comprises contacting the biomass with an ionic liquid (IL) or a (DES).

In some embodiments, the method comprises a process for the depolymerization of lignin over supported metal-nanoparticle (NP) catalyst to value-added chemicals. A supported multi-metallic NP catalysts can boost the product selectivity and overcome existing challenges including severe conditions, poor yields, and coke formation. In some embodiments, the bimetallic or trimetallic NP is immobilized on an acidic and basic support for reductive and/or oxidative depolymerization of lignin. In some embodiments, the lignin is part of a lignocellulosic biomass.

In some embodiments, the method comprises a process for the depolymerization of lignin over solid acid supported palladium catalysts. In some embodiments, the metal catalyst is a palladium supported on zirconium phosphate. Palladium catalyst is observed to be a robust catalyst for lignocellulosic lignin depolymerization to phenols and guaiacols.

In some embodiments, the catalytic efficacy of the NP catalyst and the selectivity of bond cleavage is governed by controlling the shape, size, coordination, and electron density on constituent metals. In some embodiments, the catalyst is a metal NP comprising Ni, Cu, Cr, Co, Fe, Ru, Rh, Pd, Pt, Au, Re, and/or Ir. In some embodiments, the metallic NP is immobilized on an acidic (such as ZSM-5 or SAPO-34) or basic (such as hydrotalcite or hydroxyapatite) support for reductive and oxidative chemistry, respectively. ZSM-5 (Zeolite Socony Mobil-5) is an aluminosilicate zeolite belonging to the pentasil family of zeolite. SAPO-34 is a micro pore zeolite having the formula (SiO₂)x(Al₂O₃)y(P₂O₅)z, and having a special water absorbing capacity and Bronsted acidity.

In some embodiments, the method produces equal to or less than about 20%, 15%, 10%, or 5% of char formation.

In some embodiments, some challenges can be overcome by designing and developing a tunable catalyst with easy tailoring of their activities to boost the product selectivity such as supported metallic NPs. In some embodiments, bi- and trimetallic catalysts are of considerable use particularly in catalysis as, a) formation of heteroatom bonds modifies the electronic environment, and b) geometry of the multi-metallic structure being different from the parent metals introduces strain effects. These catalysts can get better of monometallic catalysts through synergistic effects; highly active noble metals are less abundant (thus expensive), while abundant non-noble metals have limited activity.

In some embodiments, the solid acid catalyst supported palladium catalysts, such as Pd/ZrP (palladium on zirconium phosphate), achieve >90% lignin depolymerization. Herein is demonstrated the Pd/ZrP catalyzed lignin depolymerization on various lignocellulosic biomass and lignin namely technical, and ionic liquid processed biorefinery lignin (lignin obtained after one-pot pretreatment with ionic liquid and saccharification of grasses, hardwood, or softwood). The lignin valorization afforded high oil yields (>55%) based on lignin content with minimum char formation (<15%). Herein describes the product profile of depolymerized lignin using HSQC NMR and GC-MS along with the molecular weight distribution profile to observe strong effect on C—O bond cleavage as a function of temperature and time. The leftover solids (char) are also thoroughly characterized to understand the composition of the same. Finally, Pd/ZrP catalysts are reused without any significant loss in activity at least up to 4 runs.

In some embodiments, the method further comprises: (d) optionally introducing an enzyme to break down or depolymerize the cellulosic portion, including cellulose, hemicellulose, or a mixture thereof, of the solubilized, or deconstructed, or pretreated biomass, or biomass from which lignin has been obtained or released, into one or more sugar monomers, such as glucose, xylose, or a mixture thereof, and (e) introducing a microbe to the solubilized, or deconstructed, or pretreated biomass, or biomass from which lignin has been obtained or released, such that the microbe utilizes the cellulosic portion of the solubilized, or deconstructed, or pretreated biomass, or biomass from which lignin has been obtained or released, and/or one or more sugar monomers, as a carbon source to produces a biofuel or bioproduct (or chemical compound).

The present invention provides for compositions and methods described herein. In some embodiments, the compositions and methods further comprise steps, features, and/or elements described in U.S. Patent Application Publication No. 2020/0216863, hereby incorporated by reference in its entirety.

In some embodiments, the method is a one-pot method, which does not require any solid-liquid separation step. In some embodiments, the one-pot method does not require adjustment of the pH level in the one-pot composition. In some embodiments, the one-pot method does not require any dilution, or addition of water or medium. In some embodiments, the growth of the microbe occur in the same one-pot composition. In some embodiments, the IL, or mixture thereof, is renewable as it can be continuous in use.

Lignin, a complex and random cross-linked polymer of phenylpropane units, constitutes about one-third of the lignocellulosic biomass and is the most-abundant and largest bio-renewable source for aromatics. Efficient lignin valorization is of utmost importance to boost the economics of lignocellulosic biorefinery and reduce the reliance on fossil resources for aromatics/phenolics. Lignin utilization and overcoming the technical challenges associated with the complexity and heterogeneity of lignin has gained a significant attention from the scientific community in the past few years resulting in the development of various depolymerization processes. The major hurdles involve a) use of extreme conditions in chemical catalysis to afford monomers, b) poor yield and selectivity, c) severe cracking and coke formation, and d) limited efficient catalysts. For instance, reductive conditions (either H2 or a H-donor) have been frequently applied in depolymerization processes of (isolated) lignin primarily targeting the interunit ether bonds (b-O-4, a-O-4). Combining reductive catalysis with a redox catalyst such as solid acid catalyst (composed of both Bronsted and Lewis acid sites) can promote selective pathways to afford: (i) hydrogenolysis of ether bonds, (ii) removal of benzylic OH-groups, and (iii) possible removal of phenolic OH-groups.

In some embodiments, some challenges can be overcome by designing and developing a tunable catalyst with easy tailoring of their activities to boost the product selectivity such as supported metallic NPs. In some embodiments, bi- and trimetallic catalysts are of considerable use particularly in catalysis as, a) formation of heteroatom bonds modifies the electronic environment, and b) geometry of the multi-metallic structure being different from the parent metals introduces strain effects. These catalysts can get better of monometallic catalysts through synergistic effects; highly active noble metals are less abundant (thus expensive), while abundant non-noble metals have limited activity.

The catalytic efficacy of the NP catalyst and the selectivity of bond cleavage can be governed by controlling the shape, size, coordination, and electron density on constituent metals. In some embodiments, the catalyst is a metal NP comprising Ni, Cu, Cr, Co, Fe, Ru, Rh, Pd, Pt, Au, Re, and/or Ir. These metallic NPs will be immobilized on various acidic (such as ZSM-5, SAPO-34) and basic (such as hydrotalcite, hydroxyapatite) supports for reductive and oxidative chemistry, respectively.

In some embodiments, the solid acid catalyst supported palladium catalysts, such as Pd/ZrP (palladium on zirconium phosphate), achieve >90% lignin depolymerization. Herein is demonstrated the Pd/ZrP catalyzed lignin depolymerization on various lignocellulosic biomass and lignin namely technical, and ionic liquid processed biorefinery lignin (lignin obtained after one-pot pretreatment with ionic liquid and saccharification of grasses, hardwood, or softwood). The lignin valorization afforded high oil yields (>55%) based on lignin content with minimum char formation (<15%). Herein describes the product profile of depolymerized lignin using HSQC NMR and GC-MS along with the molecular weight distribution profile to observe strong effect on C—O bond cleavage as a function of temperature and time. The leftover solids (char) are also thoroughly characterized to understand the composition of the same. Finally, Pd/ZrP catalysts are reused without any significant loss in activity at least up to 4 runs.

The present invention is useful in the lignin conversion into biofuels and/or bioproducts.

The present invention described herein has the one or more of the following key points of differentiation when compared to other methods: (1) Supported multimetallic NP catalysts (lower loading). (2) Compatible with downstream processes. (3) Possible recycling of metals catalysts, including reuse in the present method. (4) Precise control over aromatic and hydrocarbon product profile. (5) Lower char formation.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Schematic of IL lignin generation and subsequent catalysis into aromatic monomers.

FIG. 2. (A) Percentage yields of liquid (blue bar), solid (green bar), gaseous (gray bar) product obtained after hydrogenolysis of [Ch][Lys]-Poplar lignin in the presence and absence of Pd/ZrP catalyst at various temperatures. (B) Yields of phenol, guaiacol, methyl guaiacol, ethyl guaiacol, propyl guaiacol, and total guaiacols (pink circle) obtained after hydrogenolysis of [Ch][Lys]-Poplar lignin in the presence and absence of Pd/ZrP catalyst at various temperatures. Bottom. Comparisons for all pairs using Tukey-Kramer HSD test.

FIG. 3. HSQC NMR showing aromatic (left), side-chain C—O—C (center), and side-chain aliphatic regions (right) of [Ch][Lys]-Poplar-Lignin (top), liquid obtained in the presence (center), and absence (bottom) of Pd/ZrP catalyst. The structural units exhibiting interunit linkages are shown as A, A′, B, and C. Regions for S, G, and H-units are marked in red, blue, and green dashed boxes in the aromatic regions, respectively.

FIG. 4. Molecular weight distribution of the starting lignin (black) and liquid obtained after treatment at 300° C. with and without catalysts—No catalyst (gray), ZrP (red), and Pd/ZrP (blue).

FIG. 5. (A) Percentage yields of liquid (blue bar), solid (green bar), gaseous (gray bar) product obtained after hydrogenolysis of [Ch][Lys]-Poplar lignin in the presence and absence of Pd/ZrP catalyst as a function of time. (B) Yields of phenol, guaiacol, methyl guaiacol, ethyl guaiacol, propyl guaiacol, and total guaiacols (pink circle) obtained after hydrogenolysis of [Ch][Lys]-Poplar lignin in the presence and absence of Pd/ZrP catalyst as a function of time. Bottom. Comparisons for all pairs using Tukey-Kramer HSD test.

FIG. 6. Resuability of Pd/ZrP catalyst. (A) Percentage yields of liquid (blue bar), solid (green bar), gaseous (gray bar) product obtained after hydrogenolysis of [Ch][Lys]-Poplar-Lignin over Pd/ZrP catalyst at 300° C. in various solvents. (B) Yields of phenol, guaiacol, methyl guaiacol, ethyl guaiacol, propyl guaiacol, and total guaiacols (pink circle) obtained after hydrogenolysis of [Ch][Lys]-Poplar-Lignin over Pd/ZrP catalyst at 300° C. in various solvents. (C) PXRD patterns of (a) ZrP, (b) Pd/ZrP, (c) Solids after 1^{st} catalytic run at 300° C. for 3 h in the presence of Pd/ZrP, and (d) solids obtained from catalytic run and after calcinination at 575° C. in air to regenerate Pd/ZrP. Bottom right. ICP-OES and CHN analysis of the lignin, catalyst, and other solids. Pd amounts in the catalyst (obtained after calcination of the solids after catalytic run or as-synthesized catalyst) was determined using ICP-OES.

FIG. 7. ANOVA for A) liquid, B) solid, and C) gas yields.

FIG. 8. Molecular weight distribution of liquid obtained after catalytic run at different temperatures in the presence of Pd/ZrP catalyst compared to the IL-based biorefinery Poplar lignin substrate (black).

FIG. 9. ANOVA for A) liquid, B) solid, and C) gas yields.

FIG. 10. Molecular weight distribution of liquid obtained after catalytic run for different time in the presence of Pd/ZrP catalyst compared to lignin substrate.

FIG. 11. (Left) Percentage yields of liquid (blue bar), solid (green bar), gaseous (gray bar) product obtained after hydrogenolysis of [Ch][Lys]-Poplar-Lignin over Pd/ZrP catalyst at 300° C. in various solvents. (Right) Yields of phenol, guaiacol, methyl guaiacol, ethyl guaiacol, propyl guaiacol, and total guaiacols (pink circle) obtained after hydrogenolysis of [Ch][Lys]-Poplar-Lignin over Pd/ZrP catalyst at 300° C. in various solvents. Reaction conditions: Lignin (0.35 g), catalyst (0.1 g), solvent (10 mL, 2:1, v/v), 300° C., 3 h, 500 rpm, N₂ (18 bar).

FIG. 12. Plausible reaction pathway of lignin hydrogenolysis over Pd/ZrP catalyst.

FIG. 13. PiMS of gaseous fraction.

FIG. 14. PiMS analysis of gas fractions for control experiments.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In some embodiments, the method uses a one-pot methodology, for example, using method steps and compositions as taught in U.S. Patent Application Publication No. 2020/0216863 (which is incorporated by reference). In some embodiments, the method further comprises heating the one-pot composition, optionally also comprising the microbe, to a temperature that is equal to, about, or near the optimum temperature for the growth of the microbe. In some embodiments, the microbe is a genetically modified host cell capable of utilizing the monomer produced as a carbon source, and produces a biofuel or bioproduct, and/or chemical compound. In some embodiments, there is a plurality of microbes.

### Ionic Liquid

Ionic liquids (ILs) are salts that are liquids rather than crystals at room temperatures. It will be readily apparent to those of skill that numerous ILs can be used in the present invention. In some embodiments of the invention, the IL is suitable for pretreatment of the biomass and for the hydrolysis of cellulose by thermostable cellulase. Suitable ILs are taught in ChemFiles (2006) 6(9) (which are commercially available from Sigma-Aldrich, Milwaukee, Wis.). Such suitable ILs include, but are not limited to, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

In some embodiments, the IL includes, but is not limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMTM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl4), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyrimidium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, fluoride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and, 10,155,735; U.S. Patent Application Publication Nos. 2004/0097755 and 2010/0196967; and, PCT International Patent Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100° C. with salts that have higher melting points referred to as molten salts. Suitable PPILs are disclosed in Greaves et al. “Protic Ionic Liquids: Properties and Applications” Chem. Rev. 108(1):206-237 (2008). PILs can be prepared by the neutralization reaction of certain Bronsted acids and Bronsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid).

Suitable IL, such as distillable IL, are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO₂-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”, Green Chem. 19(13):3152-3163 (2017), all of which are hereby incorporated by reference.

In some embodiments, the PILs are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate—[Eth][OAc]) is an effective solvent for biomass pretreatment and is also relatively cheap due to its ease of synthesis (Sun et al., Green Chem. 19(13):3152-3163 (2017)).

### Deep Eutectic Solvent (DES)

DESs are systems formed from a eutectic mixture of Lewis or Bronsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Bronsted acid and base. In some embodiments, the Lewis or Bronsted acid and base combination used is distillable.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 150° C., or 160° C., or any temperature between any two of the preceding temperatures.

In some embodiments, the DES can be one taught in WO 2018/204424 (Seema Singh et al.), which is hereby incorporated in its entirety by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing step(s) produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps described herein are continuous, the one-pot composition is in a steady state.

In some embodiments, the introducing step comprises heating the mixture comprises increasing the temperature of the solution to a value within a range of about 75° C. to about 125° C. In some embodiments, the heating step comprises increasing the temperature of the solution to a value within a range of about 80° C. to about 120° C. In some embodiments, the heating step comprises increasing the temperature of the solution to a value within a range of about 90° C. to about 110° C. In some embodiments, the heating step comprises increasing the temperature of the solution to about 100° C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram-negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. lichenformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandi; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoume; Oregon Myrtle; California Bay Laurel; Pear; Poplar (e.g. P. balsamifera, P. nigra, Hybrid Poplar (Populus x canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C4 or C3 grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inculants. In some embodiments, the ensiled biomass comprises one or more toxic compounds. In some embodiments, when ensiled biomass comprises one or more toxic compounds, the microbe is resistant to the one or more toxic compounds.

Reference cited are:
¹ Reboredo, F. H., Lidon, F., Pessoa. F. & Ramalho. J C. The fall of oil prices and the effects on biofuels. Trends Biotechnol. 34, 3-6 (2016).
² Gaurav. N. Sivasankari, S. Kiran, G S., Ninawe, A. & Selvin, J. Utilization of bioresources for sustainable biofuels: A Review. Renewable Sustainable Energy Rev 73, 205-214 (2017).
³ Gibson, L. J. The hierarchical structure and mechanics of plant materials. J. R. Soc., Interface 9, 2749-2766 (2012).
⁴ Gosselink, R. J. A., de Jong, E., Guran, B & Abacherli, A Co-ordination network for lignin: standardisation, production and applications adapted to market requirements (EUROLIGNIN). Ind. Crops Prod. 20, 121-129 (2004).
⁵ Liu, Z. H. et al., Transforming biorefinery designs with ‘plug-in processes of lignin’ to enable economic waste valorization. Nat. Commun. 12, 3912 (2021).
⁶ Wener. J., Haas, V. & Stern, T. Why can we make anything from lignin except money? Towards a broader economic perspective in lignin research. Curr. Forestry Rep. 6, 294-308 (2020).
⁷ Ren. T., Qi, W, Su, R. & He, Z. Promising techniques for depolymerization of lignin into value-added chemicals. ChemCatChem 11, 639-654 (2019).
⁸ Sun, Z., Fridrich, B., De Sant., A., Elangovan, S. & Barta, K. Bright side of lignin depolymerization: toward new platform. Chem. Rev. 118, 614-678 (2018).
⁹ Chio, C., Sain, M. & Qin, W. Lignin utilization: A review of lignin depolymerization from various aspects. Renewable Sustainable Energy Rev. 107, 232-249 (2019).
¹⁰ Rinaldi, R. et al, Paving the way for lignin valorisation: Recent advances in bioengineering, biorefining and catalysis. Angew. Chem., Int. Ed. 55, 8164-8215 (2016).
¹¹ Dimmel; D. R. & Gellerstedt, G. Chemistry of alkaline pulping, in Lignins and lignans: Advances in chemistry, eds Heitner, C. Dimmel D. R. & Schmidt, J. A. CRC Press (Taylor & Francis Group), Boca Raton, FL, ch. 10, pp 349-391 (2010).
¹² Abu-Omar, M. M. et al. Guidelines for performing lignin-first biorefining. Energy Environ. Sci. 14, 262-292 (2021).
¹³ Holbrey, J. D. & Rogers, R D. Ionic liquids in synthesis; Wasserscheid P. & Welton, T., Eds.; Wiley-VCH Verlag GmbH & Co. KGaA: Weinheim, pp 41-55 (2002).
¹⁴ Usmani, Z. et al., lonic liquid based pretreatment of lignocellulosic biomass for enhanced bioconversion. Bioresour. Technol. 304, 123003 (2020).
¹⁵ Xu. F. et al. Transforming biomass conversion with ionic liquids: process intensification and the development of a high-gravity, one-pot process for the production of cellulosic ethanol. Energy Environ. Sci. 9, 1042-1049 (2016).
¹⁶ Yao, A. et a., Can multiple ions in an ionic liquid improve the biomass pretreatment efficacy? ACS Sustainable Chem. Eng. 9, 4371-4376 (2021).
¹⁷ Sun, J. et al, One-pot integrated biofuel production using low-cost biocompatible protic ionic liquids. Green Chem. 19, 3152-3163 (2017).
¹⁸ Kim, K. H., Simmons, B. A. & Singh, S. Catalytic transfer hydrogenolysis of ionic liquid processed biorefinery lignin to phenolic compounds. Green Chem. 19, 215-224 (2017).
¹⁹ Carrozza, C. F. et al., One-pot bio-derived ionic liquid conversion followed by hydrogenolysis reaction for biomass valorization: A promising approach affecting the morphology and quality of lignin of switchgrass and poplar. Bioresour Technol. 294, 122214 (2019).
²⁰ Wang, X. C., Arai, M. Wu, Q. F., Zhang, C. & Zhao, F. Hydrodeoxygenation of lignin-derived phenolics—A review on the active sites of supported metal catalysts. Green Chem. 22, 8140-8168 (2020).
²¹ Shivhare. A. et al. Metal-acid synergy: hydrodeoxygenation of anisole over Pt/AI-SBA-15. ChemSusChem 13, 4945-4953 (2020).
²² Ma, D, Lu, S. L., Liu, X. H., Guo, Y. & Wang, Y. Q. Depolymerization and hydrodeoxygenation of lignin to aromatic hydrocarbons with a Ru catalyst on a variety of Nb-based supports. Chin. J. Catal. 40, 609-617 (2019).
²³ Xia, Q. et at, Direct hydrodeoxygenation of raw woody biomass into liquid alkanes. Nat. Commun. 7, 11162 (2016).
²⁴ Foo, G. S., Wei, D., Sholl, D. S. & Sievers, C. Role of Lewis and Bronsted acid sites in the dehydration of glycerol over niobia. ACS Catal 4, 3180-3192 (2014).
²⁵ Wang, Z., Wang, L., Jiang, Y., Hunger, tv. & Huang, J. Cooperativity of Bronsted and Lewis acid sites on zeolite for glycerol dehydration, ACS Catal. 4, 1144-1147 (2014).
²⁶ Bailleul, S. et al., A supramolecular view on the cooperative role of Bronsted and Lewis acid sites in zeolites for methanol conversion. J. Am. Chem. Soc. 141, 14823-14842 (2019).
²⁷ Marcu, C., Sandulescu, I. & Miller, J. M. Effects of the method of preparing titanium pyrophosphate catalyst on the structure and catalytic activity in oxidative dehydrogenation of n-butane. J. Moi. Catal. A 203, 241-250 (2003).
²⁸ Segawa, K., Kurusu. Y., Nakajima, Y. & Kinoshita. M. Characterization of crystalline zirconium phosphates and their isomerization activities. J. Catal 94, 491-500 (1985).
²⁹ Joshi, R. & Chudasama, U. Hydrogenation and oxidation reactions involving ruthenium supported catalysts. Ind. Eng. Chem. Res. 49, 2543-2547 (2010).
³⁰ Li, N. et at, Renewable gasoline from aqueous phase hydrodeoxygenation of aqueous sugar solutions prepared by hydrolysis of maple wood. Green Chem. 13, 91-101 (2011).
³¹ Li, N., Tompsett, G. A. & Huber, G. W. Renewable high-octane gasoline by aqueous-phase hydrodeoxygenation of C5 and C6 carbohydrates aver Pt/Zirconium phosphate catalysts. ChemSusChem 3, 1154-1157 (2010).
³² Liu, X. et al, Recent advances in the catalytic depolymerization of lignin towards phenolic chemicals: a review. ChemSusChem 13, 4296-4317 (2020).
³³ Tuteja, J., Choudhary, H., Nishimura, S. & Ebitani, K. Direct synthesis of 1,6-hexanediol from HMF over a heterogeneous Pd/ZrP catalyst using formic acid as hydrogen source. ChemSusChem 7, 96-100 (2014).
³⁴ Nakajima, K. et al, Structure and acid catalysis of mesoporous Nb₂O₅·nH₂O. Chem. Mater. 22, 3332-3339 (2010).
³⁵ Shivhare, A. Kumar, A. & Srivastava, R. An account of the catalytic transfer hydrogenation and hydrogenolysis of carbohydrate-derived renewable platform chemicals over non-precious heterogeneous metal catalysts. ChemCatChem 13, 59-80 (2021).
³⁶ Wang. D. & Astruc. D. The golden age of transfer hydrogenation. Chem. Rev. 115, 6621-6686 (2015).
³⁷ Nielsen, M. et al., Efficient hydrogen production from alcohols under mild reaction conditions. Angew Chem., Int. Ed. 123, 9767-9771 (2011)
³⁸ Weingarten, R., Tompsett, G. A., Conner, W. C. Jr. & Huber, G. W. Design of solid acid catalysts for aqueous-phase dehydration of carbohydrates: The role of Lewis and Bronsted acid sites. J. Catal. 279, 174-182 (2011).
³⁹ No monomeric lignin product was identified in the gaseous stream using photon induced mass spectrometry. The only products identified (and validated with commercial standards) were byproducts of solvent, namely, propene, dimethyl ether, and isopropyl methyl ether (FIG. S7). Propene was produced as the only (major) product in the presence of Pd/ZrP catalyst. Control experiments without lignin as substrate and with labeled isopropanol as solvent established isopropanol as the sole source of propene observed under reaction conditions (FIG. S8).
⁴⁰ Zakzeski, J., Bruijnincx, P. C. A., Jongerius A. L. & Weckhuysen, B. M. The catalytic valorization of lignin for the production of renewable chemicals. Chem. Rev. 110, 3552-3599 (2010).
⁴¹ Xu; C., Arancon, R. A. D., Labidi, J. & Luque, R. Lignin depolymerisation strategies: towards valuable chemicals and fuels. Chem. Soc. Rev. 43, 7485-7500 (2014).
⁴² Abu-Omar, M. M et al., Guidelines for performing lignin-first biorefining. Energy Environ. Sci. 14, 262-292 (2021).
⁴³ Fiege, H. et al Phenol derivatives. Ullmann's Encyclopedia of Industrial Chemistry. Weinheim: Wiley-VCH (2010).
⁴⁴ Ye, K., Liu, Y., Wu. S. & Zhuang, J. A review for lignin valorization: Challenges and perspectives in catalytic hydrogenolysis. Ind. Crops Prod. 172, 114008 (2021).
⁴⁵ Madon, R. J. & O'Connell, J. P. Catalytic hydrogenation of cyclohexene: Part II. Liquid phase reaction on supported platinum in a gradientless slurry reactor. AlChE J. 24, 904-911 (1978).
⁴⁶ Bassett, J. M., Dalmai-Imelik, G., Primet, M. & Mutin, R. A study of benzene hydrogenation and identification of the adsorbed species with PtAl2O3 catalysts. J. Catal. 37, 22-36 (1975).
⁴⁷ Wilson, O. M., Knecht, M. R. Garcia-Martinez, J. C. & Crooks, R. M. Effect of Pd nanoparticle size on the catalytic hydrogenation of allyl alcohol. J. Am. Chem. Soc. 128, 4510-4511 (2006).
⁴⁸ Slulter, A. et al., Determination of structural carbohydrates and lignin in biomass. Golden, CO: National Renewable Energy Laboratory (NREL) Analytical Procedures. TP-510-42618 (2011).
⁴⁹ Sheps, L., Antonov, I. & Au, K. Sensitive mass spectrometer for time-resolved gas-phase chemistry studies at high pressures. J. Phys. Chem. A 123, 10304-10814 (2019).
⁵⁰ Guerra, A., Lucia, L. A. & Argyropoulos, D. S. Isolation and characterization of lignins from Eucalyptus grandis Hill ex Maiden and Eucalyptus globulus Labill. by enzymatic mild acidolysis (EMAL). Holzforschung 62, 24-30 (2008).

### Example 1

Funneled Depolymerization of Ionic Liquid-Based Biorefinery “Heterogeneous” Lignin into Guaiacols Over Reusable Palladium Catalyst

The efficient utilization of lignin, the only direct source of renewable aromatics, into value-added renewable chemicals is an important step towards sustainable biorefinery practices. Nevertheless, owing to the random heterogeneous structure and limited solubility, lignin utilization has been primarily limited to burning for energy. The catalytic depolymerization of lignin has been proposed and demonstrated as a viable route for lignin utilization, however, low yields and poor selectivity of products, high char formation, and limited to no recycling of transition-metal-based catalyst demands attention to enable practical-scale lignocellulosic biorefineries. In this study, we demonstrate the catalytic depolymerization of ionic liquid-based biorefinery poplar lignin into value-added chemical products over a reusable zirconium phosphate supported palladium catalyst. The essence of the study lies in the high conversion (>80%), minimum char formation (7-16%), high yields of guaiacols (up to 60 g/L), and catalyst reusability. Both solid residue, liquid stream, and gaseous products were thoroughly characterized using ICP-OES. PXRD, CHN analysis, GC-MS, GPC, and 2D NMR to understand the hydrogenolysis pathway.

We hypothesize that the use of air, water, and heat stable ZrP with a tunable Bronsted/Lewis acid site ratio could not only facilitate higher conversion and low char formation but also facilitate easy recycling of the catalyst. Herein, we explore the scope of ZrP as a solid acid catalyst support in the hydrogenolysis of IL-based biorefinery lignin. ZrP supported palladium catalysts have been reported to be most stable and versatile for high temperature organic transformations in the presence of a hydrogen donor.

### Results and Discussion

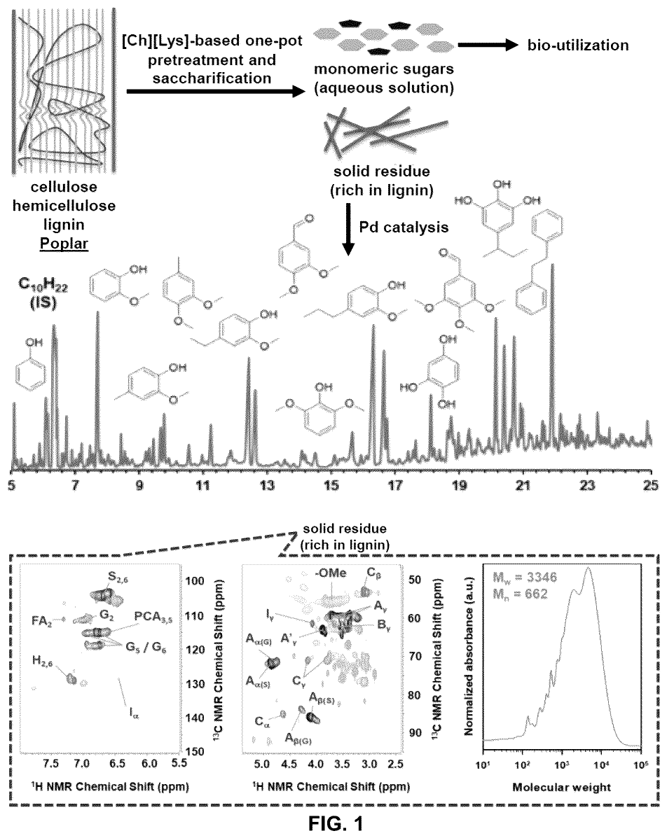

A one-pot biomass conversion technology including pretreatment with aqueous cholinium lysinate ([Ch][Lys]) and saccharification was employed to generate IL-based biorefinery lignin from poplar biomass as reported previously by our research group (15, 16, 17). This recovered solid reside was composed of 42.3% lignin along with 19.3% and 3.8% glucan and xylan, respectively. This lignin-rich solid residue was used to study the catalytic depolymerization of IL-based biorefinery lignin over zirconium phosphate supported palladium catalyst as discussed vide infra (FIG. 1).

In order to understand the catalytic depolymerization of the lignin, the solids recovered after one-pot pretreatment and saccharification (FIG. 1) were thoroughly characterized to understand the structure of phenylpropanoid units and interunit linkages, molecular weight distribution, and elemental composition using HSQC NMR, GPC and elemental analysis, respectively. Based on the HSQC analysis of the solid residue, syringyl (S) units dominated with about 69% distribution with about 25% guaiacyl (G) and about 6% hydroxyphenyl (H) units. This is consistent with the general phenylpropanoid distribution in lignin of a hardwood like poplar (32). In contrast to the technical lignin, a high number of C—O—C interunit linkages i.e., β-O-4 (about 54%) and 4-O-5 (about 5%) were present as determined by HSQC NMR. Among C—C bonds, about 39% β-β and about 2% β-5 linkages held together the three phenylpropanoids. Weight average (M_{w}) and number average (Mₙ) molecular weights of the IL-based biorefinery poplar lignin ([Ch][Lys]-Poplar lignin) was observed to be 3346 and 662, respectively, with a polymer dispersity index of 5.02.

To study the hydrogenolysis of [Ch][Lys]-Poplar lignin, zirconium phosphate (ZrP) supported palladium catalyst was prepared following a previous report (33). ZrP is a well-established solid acid catalyst with a tunable Bronsted/Lewis acid site ratio (28, 31). ZrP was synthesized by stirring aqueous solution of zirconyl chloride and sodium dihydrogen phosphate to afford Zr:P ratio of 0.5 (see Experimental for details). The obtained material was treated with dilute nitric acid to increase the Bronsted acid sites of a solid acid catalyst and thereby the Bronsted/Lewis acid site ratio (34). An enhanced number of Bronsted acid sites will result in strong interactions with the secondary alcohol group in lignin forming stable carbenium ions, while Lewis acid sites forms a stable intermediate species through chemisorption (24, 25, 26). Palladium was adsorbed onto the acid treated ZrP by simply stirring ZrP in an aqueous solution of palladium nitrate. The solids obtained were then calcined in air to obtain 5 wt % Pd/ZrP. The crystalline phase of the catalyst was identified and compared to the literature (33). The actual palladium loading was measured using ICP-OES and was found to be 4.96%.

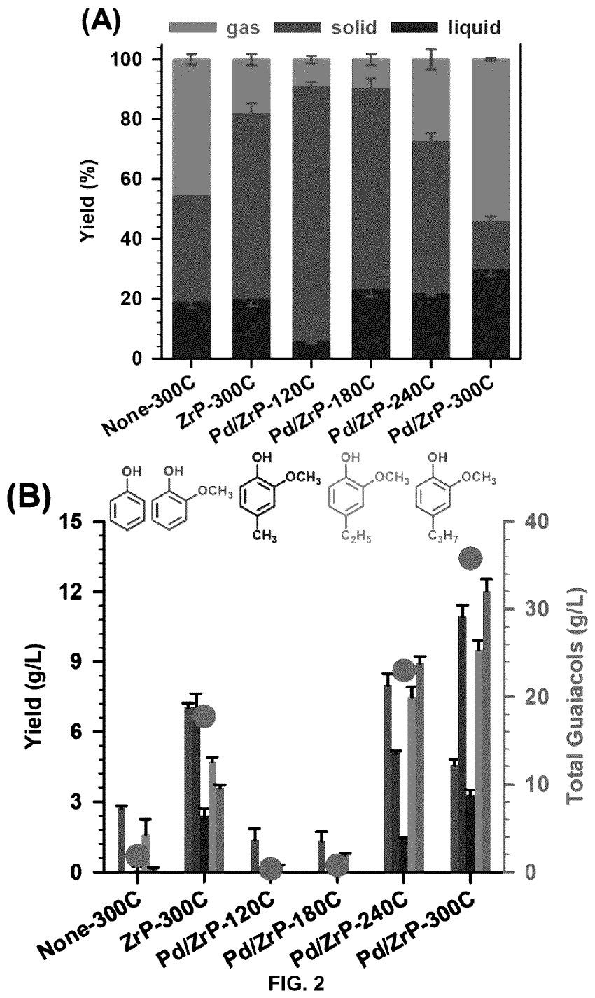

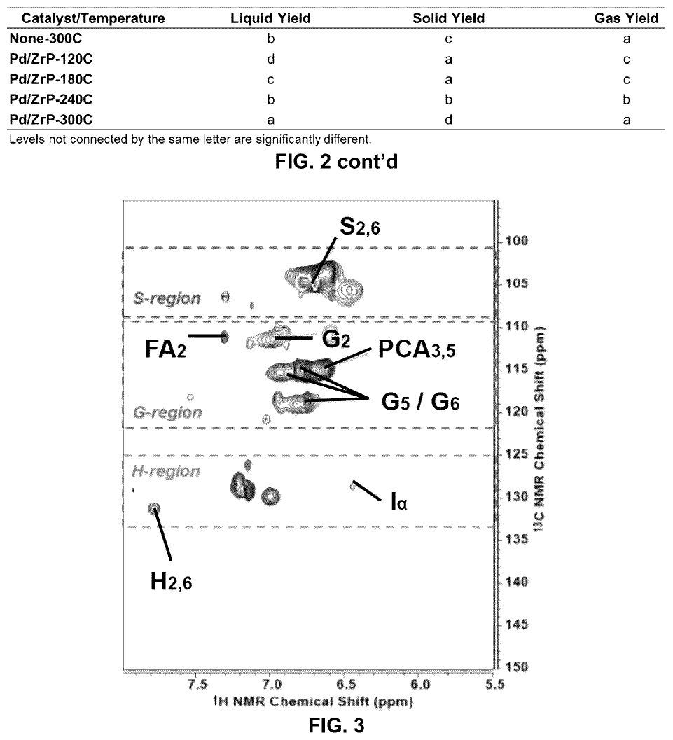

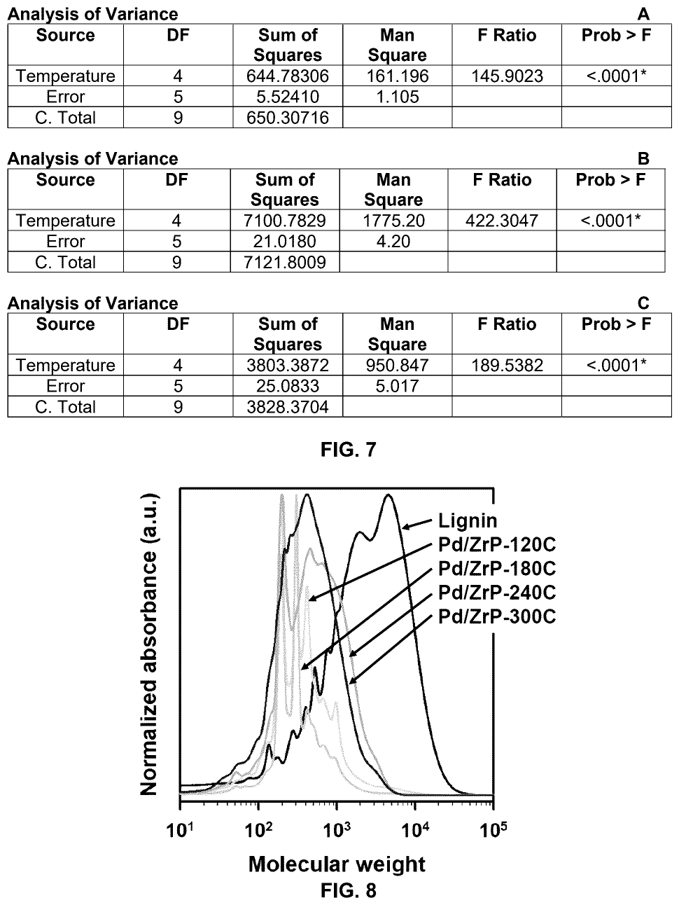

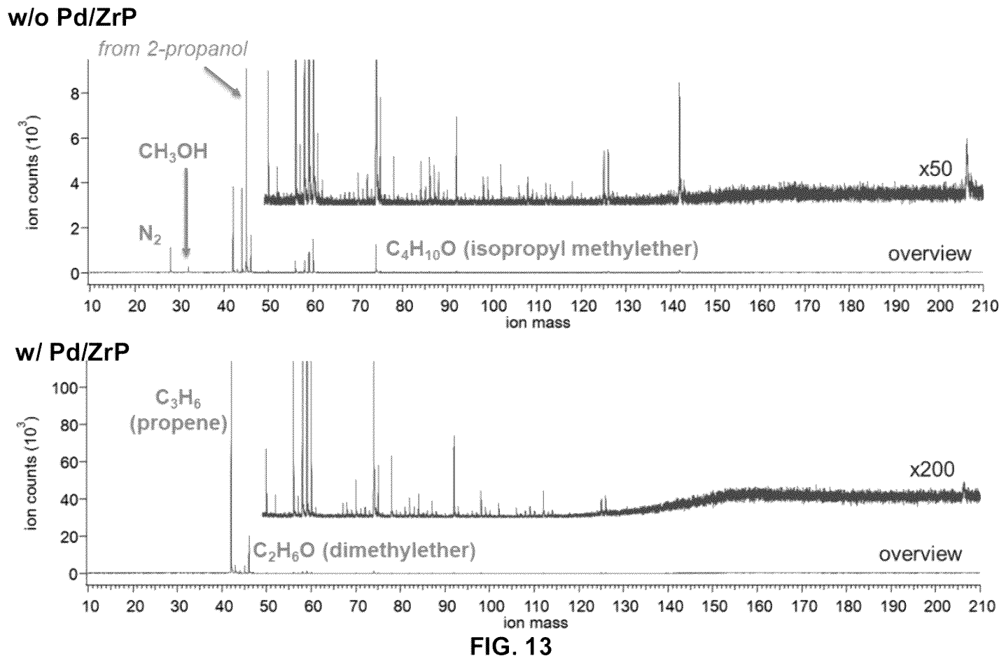

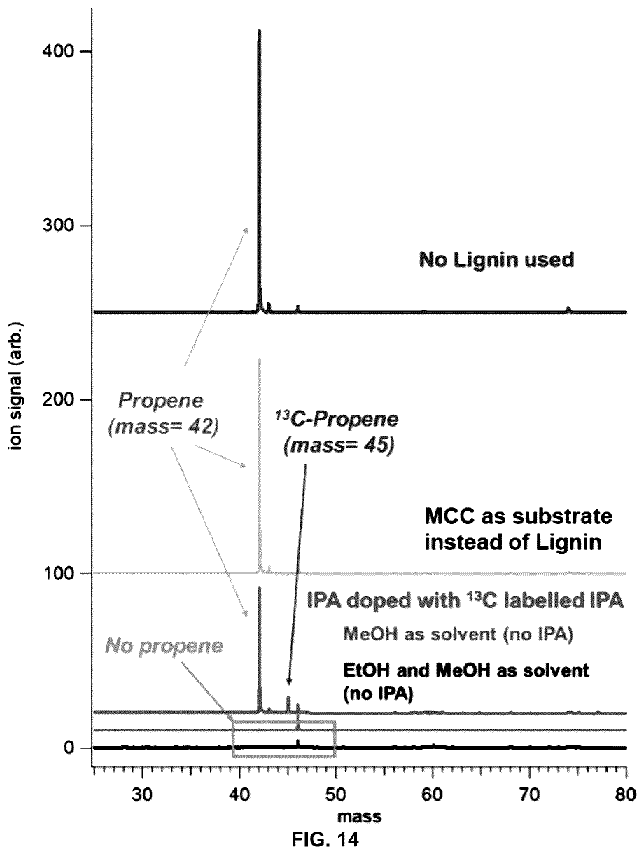

The catalytic hydrogenolysis of lignin over Pd/ZrP was performed by heating lignin and catalyst in a hydrogen donor solvent under an inert atmosphere. FIG. 2 summarizes the results for hydrogenolysis of lignin over Pd/ZrP as a function of temperature in the presence of isopropanol-methanol solvent systems that also serve as hydrogen-donor. Alcohols are reported to release hydrogen in the presence of metal catalysts and have been considered as safe and sustainable hydrogen source over pressurized hydrogen gas (35, 36, 37). Based on the previous reports, a high Bronsted/Lewis acid ratio favors the hydrogenolytic C—O bond cleavage, whereas a transition metal like palladium leads hydrogenation of the intermediates after activating hydrogen release from the hydrogen donors employed (33, 38). The mass yields of reaction products that is liquid, solid, and gas after lignin hydrogenolysis over Pd/ZrP, ZrP, and no catalyst (none) are shown in FIG. 2 (Panel A). The vacuum dried IPA-methanol soluble depolymerized fraction was weighed to obtain the mass of the liquid, whereas the insoluble residue was termed as solid (unreacted lignin and/or char). An increase in the liquid fraction yield (5.4%<22.5% about 21.3%<29.3%) and a subsequent decrease in the solid yield (85.3%>67.6%>51.1%>16.2%) was observed as the temperature was increased from 120° C. to 300° C. at 60° C. increments. Elemental analysis of the solids revealed limited conversion of lignin at lower temperatures (120-240° C.) in the presence of Pd/ZrP with a C/O ratio of <2.1 compared to substrate with a C/O ratio of 1.18. The increase of reaction temperature to 300° C. in the presence of the catalyst afforded high conversion with only 16% solid residue having a C/O ratio of 12. A maximum liquid yield of 29.3% was obtained based on the substrate (i.e., one-pot IL-based Poplar lignin) employed at 300° C., which corresponds to 69.3% based on the actual lignin content in the TL-based Poplar lignin. To demonstrate and understand the superior performances of the Pd/ZrP catalyst under applied catalytic conditions, control experiments with only ZrP and without catalyst were performed. 18.5% of liquid was obtained by simply heating substrate in solvent at 300° C. in the absence of the catalyst and could be improved to about 21% in the presence of ZrP. The solids yield without catalyst was 35.6% with the total C/O ratio of 4.56. Interestingly, solids were obtained in larger quantities with only ZrP as catalyst at 300° C. compared to with or without Pd/ZrP. about 55% reduction in char amounts was observed in the presence of Pd/ZrP as compared to non-catalytic run. The lower carbon balance in solid and liquid fractions at higher temperatures were observed with mostly due to the absence of any detectable/identifiable gaseous products (39). (No monomeric lignin product was identified in the gaseous stream using photon induced mass spectrometry. The only products identified (and validated with commercial standards) were byproducts of solvent, namely, propene, dimethyl ether, and isopropyl methyl ether (FIG. 13). Propene was produced as the only (major) product in the presence of Pd/ZrP catalyst. Control experiments without lignin as substrate and with labeled isopropanol as solvent established isopropanol as the sole source of propene observed under reaction conditions (FIG. 14).) ANOVA results (FIG. 7) illustrate that there is a significant (p<0.0001) difference between the means of temperature for liquid, solid, and gas yield. Additionally, multiple comparison analysis for liquid, solid, and gas yield with respect to temperature were obtained (Table in FIG. 2). Results from the Tukey-Kramer HSD test shows that liquid yield without catalyst at 300° C. is not significantly different from liquid yield with catalyst at 240° C., while the liquid yields in the absence of catalyst at 300° C. is significantly different from the liquid yields at all other temperatures i.e., 120, 180, and 300° C. In the case of solid yield without catalyst at 300° C. is significantly different from all other temperatures. Similarly, gas yield in the absence of the catalyst at 300° C. is not significantly different from gas yield with catalyst at 300° C., while it is significantly different from all other temperatures.

In addition to the yield of liquid, significance of Pd/ZrP in lignin hydrogenolysis was realized from GC-MS analysis of the liquid fraction, establishing the importance of acid support (ZrP) for C—O bond cleavage and palladium for hydrogenation (FIG. 2, Panel B). Among various products observed as per the NIST library, Table 1 enlists the chemical name, structure, and retention times of the molecules validated against the MS profile of the commercial standards. Traces of phenol and guaiacols (i.e., guaiacol, 4-methylguaiacol, 4-ethylguaiacol, and 4-propylguaiacol) were observed without catalyst which were marginally improved to obtain about 19 g/L total guaiacols in the presence of only ZrP at 300° C. For Pd/ZrP, in agreement to the liquid yield, hardly any monoaromatics were observed at 120 and 180° C. Nevertheless, further increase to 240° C., about 8 g/L phenol and about 25 g/L total guaiacols were obtained in the presence of Pd/ZrP. The yields for total guaiacols were further improved to about 38 g/L at 300° C. It should be highlighted that under the GC analysis conditions, traces of products derived from the sugar component of the IL-Poplar lignin were observed below 6 minutes. Notably, in contrast to typical high temperature hydrogenolysis of complex biopolymers such as lignin that forms arrays of monomeric and oligomeric products, only a few aromatic products were observed over Pd/ZrP catalyst. Also, no molecules corresponding to the over reduction of aromatics were observed in the GC-MS profile even in traces, this could be possibly because of the use of hydrogen donor instead of hydrogen gas for a severe hydrogenolysis (40, 41, 42). The guaiacols obtained in this case can be further utilized (without separation) as a raw material for synthesis in number of industries, namely food and fragrance, pharmaceutical, personal care, and industrial cleaning (43).

TABLE 1
CHN analysis of lignin and solids obtained after
reaction as a function of temperature.

| Solids | % C | % H | % N | % O |
|---|---|---|---|---|
| Lignin | 49.44 | 6.67 | 1.86 | 42.03 |
| Pd/ZrP-120 C. | 51.70 | 6.63 | 1.63 | 39.87 |
| Pd/ZrP-180 C. | 55.09 | 7.13 | 1.55 | 35.63 |
| Pd/ZrP-240 C. | 60.05 | 6.08 | 4.17 | 28.85 |
| Pd/ZrP-300 C. | 81.83 | 7.33 | 3.50 | 6.80 |
| None-300 C. | 74.85 | 5.98 | 2.75 | 16.42 |

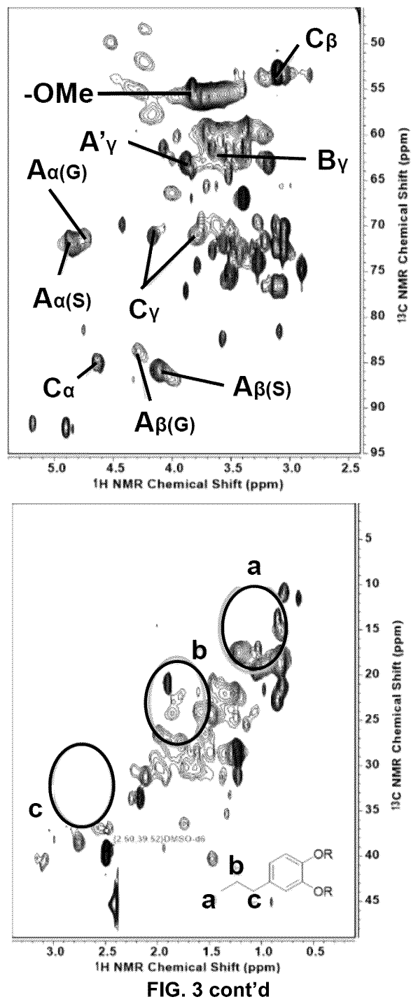

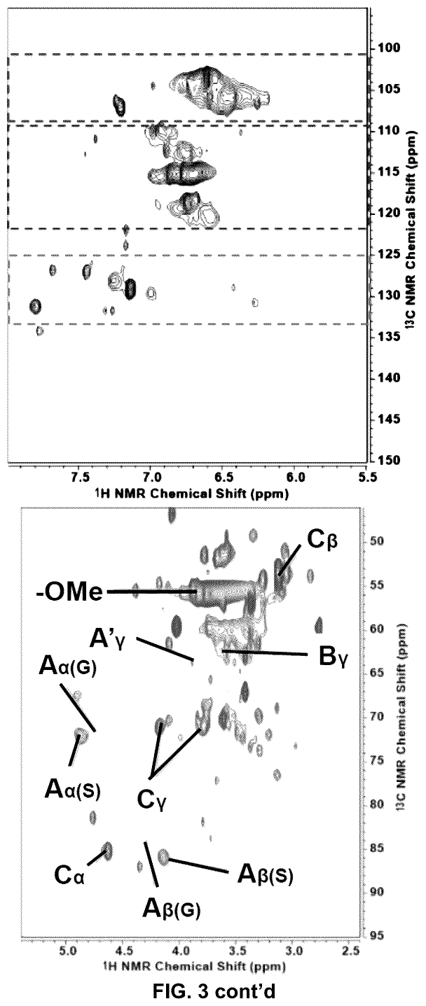

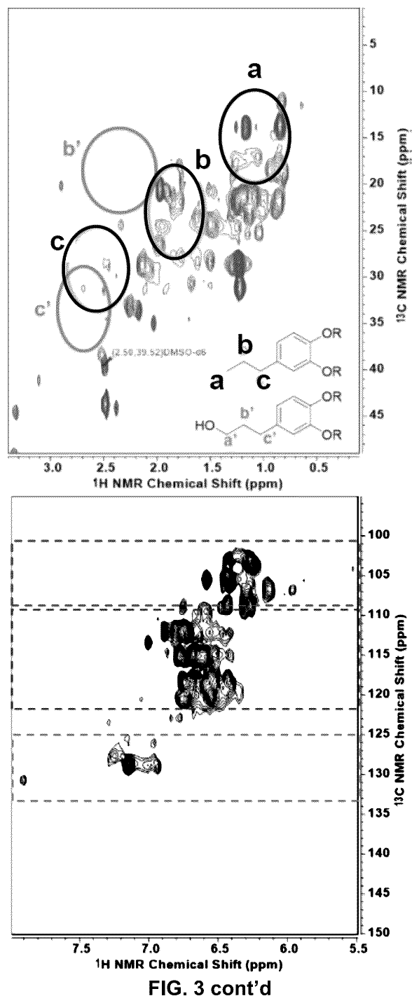

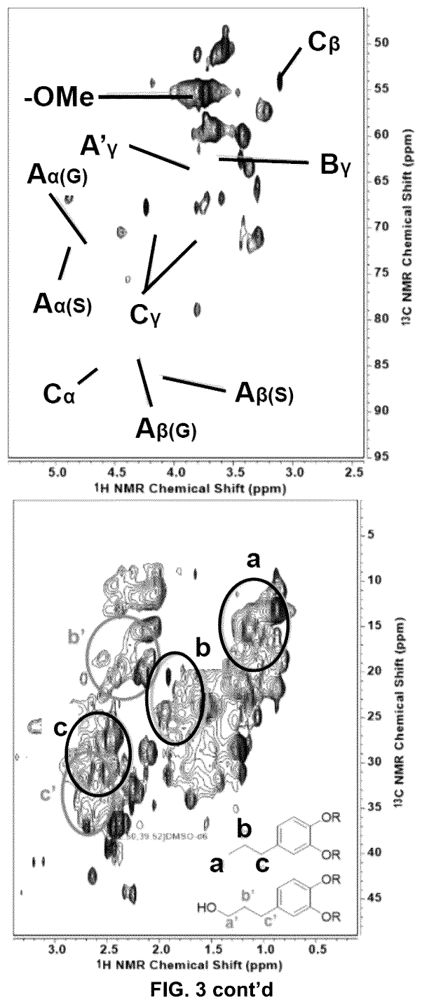

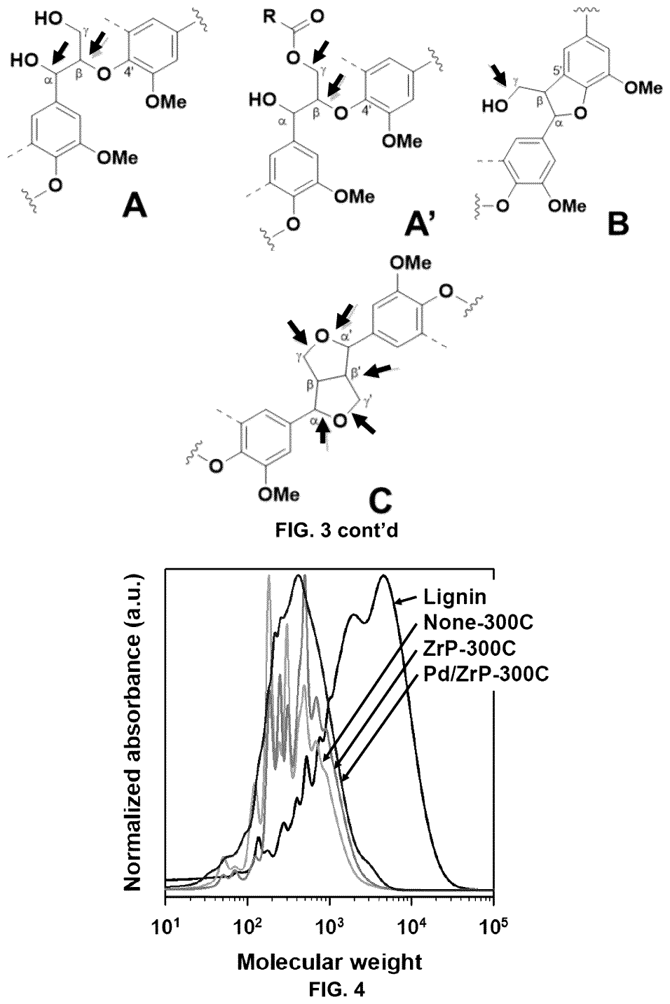

Furthermore, HSQC NMR was used to study the structural and molecular compositions of the liquid obtained after hydrogenolysis reaction. FIG. 3 compares the aromatic, side-chain C—O—C, and side-chain aliphatic regions of [Ch][Lys]-Poplar lignin and the liquid obtained after Pd/ZrP catalysis at 300° C. The interunit C—O—C and C—C linkages contained in lignin substrate shown in A, A′, B, and C structural units were absent from the liquid after the Pd/ZrP catalysis indicating the bond cleavage. This was further supported by the diffused aromatic signals of S and G units for various monomeric products as observed in a typical GC spectrum (FIG. 1). Evidently, an increase in the intensity of the signals in the G-region (with a simultaneous decrease in the S-region) indicates the demethoxylation of S-units to G-units as also seen in the GC-MS product profile. The cleavage of C—O—C linkage would result in the increase in the aliphatic side chain signals as observed in both HSQC regions marked by a, b, c, b′, and c′ for the α, β, and γ-protons of phenyl, guaiacyl, or syringyl products (FIG. 2, Panel B). The yellow arrows in the structural units indicate the bonds cleaved and/or impacted in the presence of Pd/ZrP catalyst. Evidently from the HSQC, while C—O—C linkages such as β-O-4 and 4-O-5 were completely cleaved, C—C linkages such as β-β were not impacted significantly resulting in some solid residues. For the catalytic run without catalyst, lignin was found to be solubilized (partially) under the reaction conditions but was not depolymerized as the all relevant linkages were observed to be intact.

To understand the effect of catalyst on molecular weight distribution (MWD) of lignin and to identify any larger molecular weight aromatics, GPC analysis was conducted on the liquid obtained after hydrogenolysis with ZrP, Pd/ZrP and without catalyst and compared with the lignin substrate as shown in FIG. 4. As depicted from the figure, liquid obtained in the absence of catalysts, and the presence of ZrP and Pd/ZrP has a lower MW than the starting substrate. M_{w} of the liquid without Pd/ZrP, with ZrP, and Pd/ZrP were observed to be 398, 439, and 411, respectively in contrast to 3346 for the starting material. Similarly, Mₙ of the liquid without Pd/ZrP, with ZrP, and Pd/ZrP were observed to be 153, 212, and 178, respectively. Based on the molecular weight distribution along with the product profiles in the GC-MS and HSQC NMR, it can be concluded that Pd/ZrP catalyst had a significant role in depolymerization of lignin into monoaromatics in higher concentrations. The MWD of the liquid as a function of temperature in the presence of Pd/ZrP catalyst marked an increase in the product intensity and distribution complementing the GC-MS data (FIG. 8).

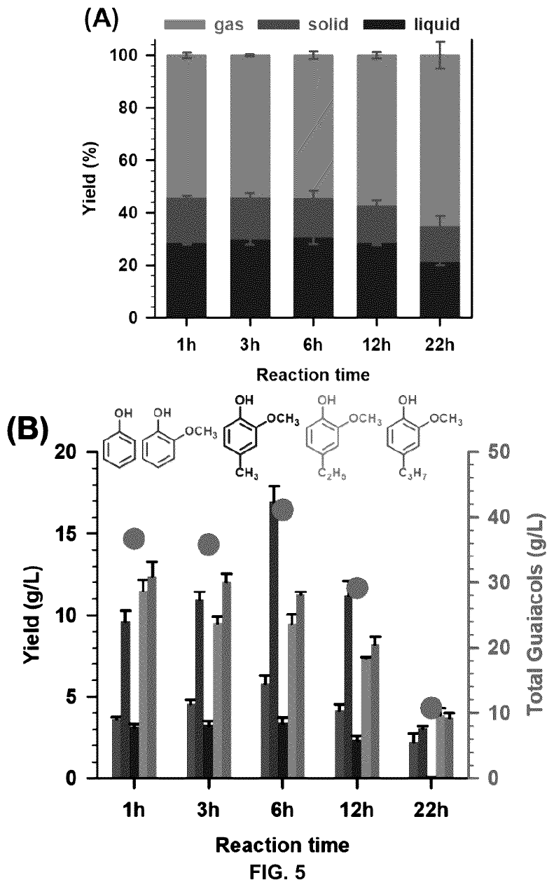

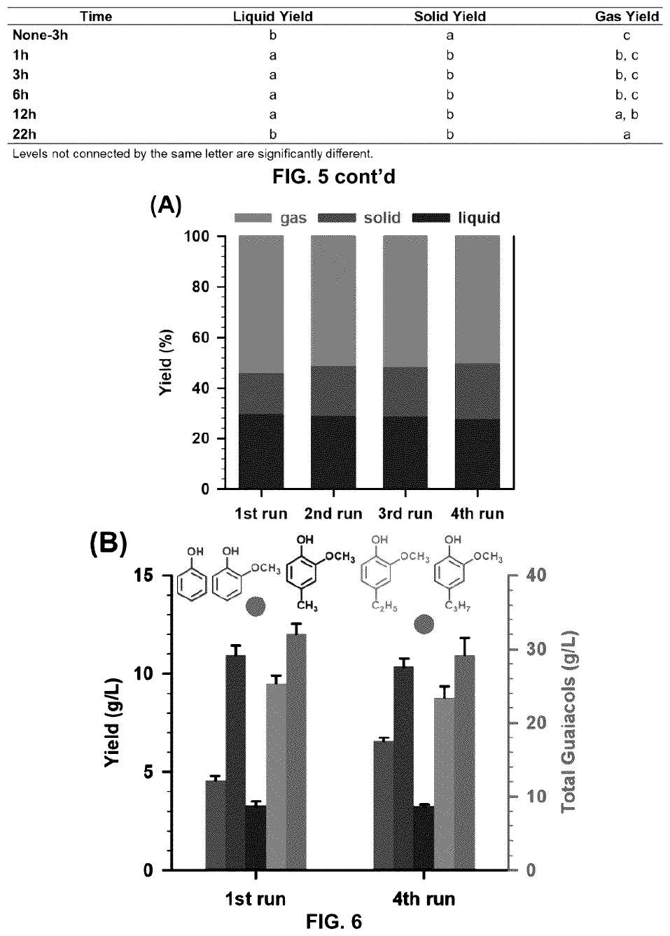

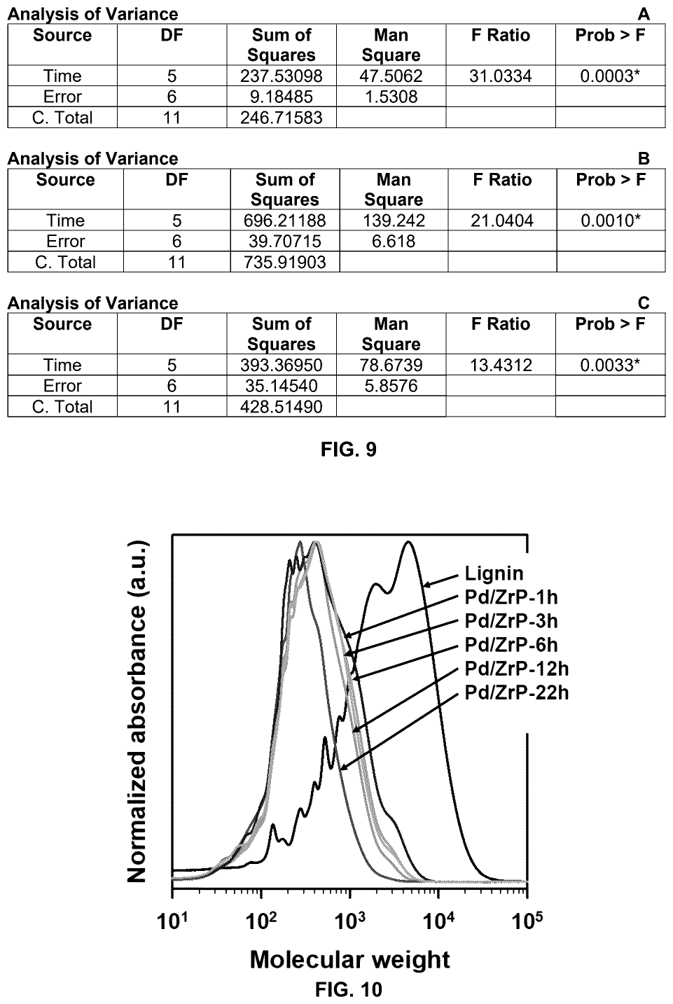

The progress of the reaction and change in the product profile was monitored by running the catalytic hydrogenolysis over Pd/ZrP for different time i.e., 1, 3, 6, 12, and 22 h (FIG. 2, Panel A). First, the mass yield of liquid, solid, and gaseous fractions were analyzed to track the progress of the reaction. The yield of liquid increased marginally as the time was increased from 1 h (about 28.0%) to 3 h (about 29.3%), from 3 h to 6 h (about 30.0%), and then to 12 h (about 28.0%). Solid yield was also similar and in the range of 14-17% for 1, 3, 6, and 12 h. With further increase in reaction time, the liquid yield dropped to 20.7% with a 13.9% solid yield. ANOVA results (FIG. 9) illustrate that there is a significant (p<0.05) difference between the means of time for oil, solid, and gas yield. Table in FIG. 5 shows the multiple comparison analysis for oil, solid, and gas yield with respect to time via Tukey-Kramer HSD test. Obtained results for the oil yield depicts no significant difference between without catalyst at 3 h and with Pd/ZrP catalyst at 22 h, while all other time points are significantly different from the catalytic run without catalyst. Interestingly, solid yield without a catalyst is significantly different from all time points. Additionally, gas yield for no catalyst is significantly different from 12 h and 22 h time points, while no catalyst is not significantly different from other time points.

Quantification of major products, phenol and guaiacols at different reaction times depicted the enhanced C—C bond cleavage in the form of demethylation of alkylguaiacols into guaiacol (FIG. 2, Panel B). The total guaiacol yields were in the range of 36-40 g/L for 1, 3, and 6 h. The yields for total guaiacols decreased on continuing the reaction for 12 and 22 h yielding about 30 and 10 g/L total guaiacols, respectively. The MWD plot for the time dependent lignin hydrogenolysis demonstrated narrowing of the curve with the increase in reaction time from 1 to 22 h (FIG. 10).

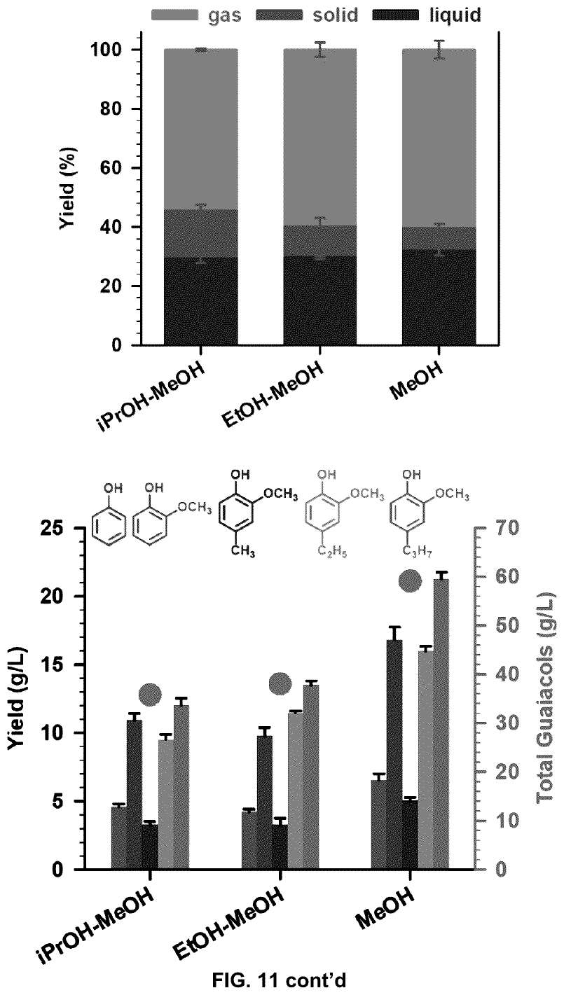

The conversion of lignin and yields of phenol and guaiacol were further improved by changing solvent system from isopropanol-methanol (IPA-MeOH) mixture to ethanol-methanol (EtOH-MeOH) mixture to only MeOH (see FIG. 11). Although, the EtOH-MeOH mixture afforded similar liquid yield (29.6%) as that of the IPA-MeOH mixture (29.3%), the solid yields were decreased significantly from 16.2% for IPA-MeOH system to 10.4% for EtOH-MeOH solvent system. The yields for phenol and guaiacol were similar for both IPA-MeOH and EtOH-MeOH systems. When only MeOH was used as the solvent and hydrogen donor for lignin hydrogenolysis over Pd/ZrP, the amount of char was reduced to as low as 7.6%. The reduced amounts of char had a direct influence on the guaiacols yield to afford about 60 g/L of total guaiacols compared to about 38 g/L in IPA-MeOH.

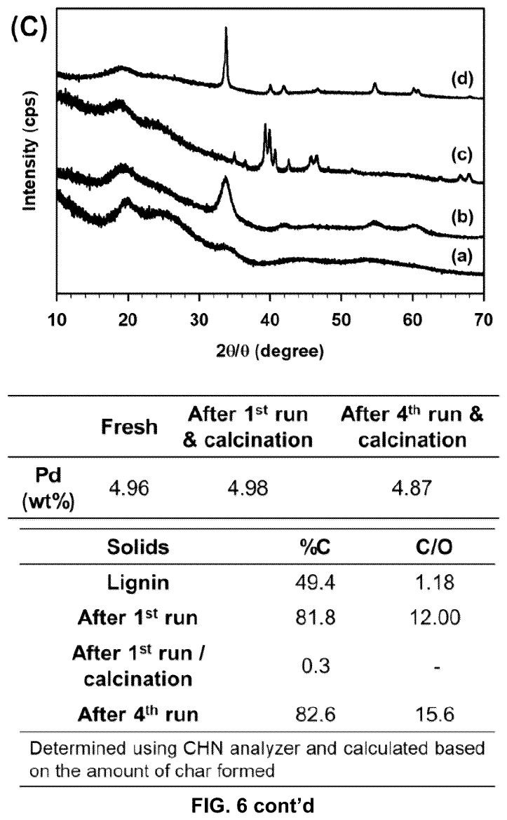

One of the major technical bottlenecks in catalytic hydrogenolysis of lignin into monoaromatics is the catalyst stability and robustness (44). Owing to the use of high temperature and pressure, most catalysts employed for hydrogenolysis deactivates after catalytic run—either because of coking/lignin deposition, surface saturation, or catalyst leaching/sintering. These could limit the application and deployment of catalytic hydrogenolysis of lignin. Nevertheless, catalyst recyclability and reusability has been an open question for the researchers around the globe. Thereby, the reusability of the Pd/ZrP was investigated next (FIG. 6). The liquid fraction was first analyzed for any leached catalyst component including Pd, Zr, and P. The ICP-OES analysis confirmed that neither the supported Pd nor the assembled Zr and P leached during the catalytic hydrogenolysis of lignin under our reaction conditions on the ppm order. The dry solids obtained after separating the liquid fraction and washing with methanol contained both catalyst and unreacted lignin and/or char. The carbon content of the solids after the 1^{st} catalytic run at 300° C. was found to be 81.8% with a C/O ratio of 12 compared to the 49.4% C content and a C/O ratio of 1.2 in the lignin substrate as determined by elemental analysis. PXRD patterns of these solids identified metallic Pd in contrast to the ionic/oxide Pd species in the fresh catalyst (FIG. 6, Panel C). As anticipated, the reducing condition during the hydrogenolysis reaction reduced the oxidized Pd species to metallic Pd. No significant phases were determined by PXRD other than amorphous ZrP. To regenerate the oxide phase of Pd, solids were calcined in air at 575° C. for 6 h. Calcination of the solids in air resulted in the oxidation of char/lignin (C content reduced to 0.3%) deposited on the catalyst rendering larger crystallite size of ionic/oxide Pd species. This is in agreement with the previous report on Pd/ZrP.^{Error! Bookmark not defined.} It is important to highlight that the hydrogenation activity of any transition metal is not dependent directly on its crystallite size as reported earlier (45, 46, 47) and thereby no significant difference was observed in the catalytic activity of Pd/ZrP in the present case. The regenerated catalyst (after calcination) was used for lignin hydrogenolysis for another three times with a calcination cycle in between two catalytic runs. ICP-OES analysis on the regenerated catalyst after 1^{st} and 4^{th} runs ruled out any Pd loss from the active catalyst during lignin hydrogenolysis. The mass yield for various fractions were recorded and are shown in FIG. 6 (Panel A). The yield of the liquid fraction was similar even after 4 cycles, with a slight increase in solid yield with each run. Quantification of phenol and guaiacol products suggested no significant loss of catalytic activity, establishing reusability and robustness of the Pd/ZrP catalyst in lignin hydrogenolysis (FIG. 6, Panel B).

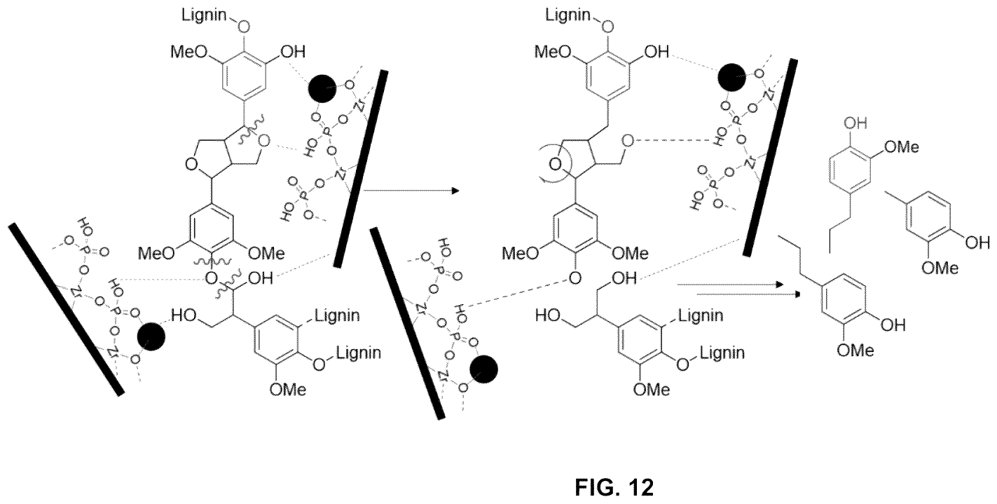

Based on the available literature and observed experimental results, the hydrogenolysis of lignin is anticipated to proceed through following steps: 1) chemisorption/interaction of lignin and catalyst, 2) formation of stable intermediate species, 3) C—O bond cleavage through dehydration or deoxygenation on Bronsted acid sites, and 4) hydrogenation of C—O and C—C bonds on metal sites. A representative plausible reaction pathway of lignin hydrogenolysis into phenol and guaiacols over Pd/ZrP catalyst is sketched as FIG. 12. Rigorous experiments are required to gain a clear understanding of the mechanistic pathway of lignin hydrogenolysis reaction.

### CONCLUSIONS

Commercial biorefineries must be able to convert the majority of components present in lignocellulose, including sugars and lignin-derived intermediates, into biofuels and bioproducts. We have developed a catalytic hydrogenolysis of TL-based biorefinery lignin over reusable Pd/ZrP catalyst without compromising fermentable sugar yields. Oil yields as high as about 70% based on initial lignin content containing up to 60 g/L of guaiacols were achieved with minimum char formation (about 15%). The analysis of the product streams using various analytical techniques suggested extensive C—O bond cleavage over the acidity of ZrP followed by hydrogenation using Pd in the presence of hydrogen source. This study demonstrates a potential catalytic conversion of IL-based biorefinery lignin and rigorous models to understand the reaction pathway, catalytic sites, environmental and economic impact are required to enable overall sustainability and economic viability.

### Experimental

### Materials

All materials were used as supplied unless otherwise noted. Water was deionized, with specific resistivity of 18 MQ cm at 25° C., from Purelab Flex (ELGA, Woodridge, IL). Cholinium lysinate ([Ch][Lys]) was procured from Proionic GmbH (Raaba-Grambach, Austria). J. T. Baker, Inc. (Phillipsburg, NJ) supplied hydrochloric acid. Sodium hydroxide pellets (>97%) and analytical standard grade glucose and xylose for calibration were obtained from Sigma-Aldrich (St. Louis, MO). Biomass studied here was Poplar. The biomass was dried for 24 hours in a 40° C. oven. Subsequently, it was knife-milled with a 2 mm screen (Thomas-Wiley Model 4, Swedesboro, NJ). Commercial cellulase (Cellic® CTec3) and hemicellulase (Cellic® HTec3) mixtures were provided by Novozymes, North America (Franklinton, NC).

### Generation of [Ch][Lys]-Poplar Lignin

[Ch][Lys]-based biomass pretreatment and subsequent saccharification was performed in a one-pot configuration in a 1 L Parr 4520 series Bench Top reactor (Parr Instrument Company, Moline, IL). Typically, 2 mm poplar samples, IL, and water were mixed in a 1.5:1:7.5 ratio (w/w) (15 wt % biomass loading) in the Parr vessel. The prior mixing is critical and ensures homogeneous mixtures of biomass, IL, and water. The slurry was pretreated for 3 hours at 140° C. with stirring at 80 rpm powered by process (Parr Instrument Company, model: 4871, Moline, IL) and power controllers (Parr Instrument Company, model: 4875, Moline, IL) using three-arm, self-centering anchor with PTFE wiper blades. After 3 hours, the pretreated slurry was cooled down to room temperature by removing the heating jacket. The pH of the cold pretreated mixture was noted (pH about 10.5) and adjusted to 5 with concentrated hydrochloric acid. Enzymatic saccharification was carried out at 50° C. for 72 hours at 80 rpm using enzyme mixtures Cellic® CTec3 and HTec3 (9:1 v/v) at a loading of 10 mg protein per g biomass. After 72 hours, the slurry was centrifuged and washed multiple times with DI water to remove any residual sugar (washed until the pH of the washing was neutral). The washed material was freeze dried to obtain [Ch][Lys]-Poplar lignin and was characterized using PXRD, HSQC, CHNS, and GPC. The lignin content was analyzed using NREL two-step acid hydrolysis laboratory analytical protocols (48). All compositional analysis experiments were conducted in duplicate. Briefly, 200 mg of biomass and 2 mL of 72% sulfuric acid (H₂SO₄) were incubated at 30° C. while shaking at 200 rpm for 1 h. The slurry was diluted to 4% H₂SO₄ with 56 mL of DI water for a secondary hydrolysis at 121° C. for 1 h. The reaction was quenched by cooling down the flasks before removing the solids by filtration. Filtrate was used to determine glucan, xylan, and acid soluble lignin (ASL) composition while the acid insoluble lignin (AIL) and ash amounts were calculated from the solid residue. Glucose and xylose concentrations were determined from the filtrate using an Agilent HPLC 1260 infinity system (Santa Clara, California, United States) equipped with a Bio-Rad Aminex HPX-87H column and a Refractive Index detector at 35° C. An aqueous solution of sulfuric acid (4 mM) was used as the eluent (0.6 mL min⁻¹, column temperature 60° C.). The amount of glucan and xylan was calculated from the glucose and xylose content multiplied by the anhydro correction factors of 162/180 and 132/150, respectively. ASL was estimated by measuring the UV absorption of the acid hydrolysis supernatant at 240 nm using a UV-Vis spectrophotometer (Nanodrop 2000, Thermo Scientific, USA). AIL was quantified gravimetrically from the solid after heating overnight at 105° C. (to obtain the weight of AIL+ash) and then at 575° C. for at least 6 h (corresponding to the weight of ash).

### Synthesis of Zirconium Phosphate (ZrP)

Zirconium phosphate (ZrP, Zr/P=0.5) was prepared as reported elsewhere (31). ZrP was prepared by dropwise addition of 100 mL 0.1 M zirconyl chloride (ZrOCl₂·8H₂O) into a stirring 100 mL aqueous solution of 0.2 M sodium dihydrogen phosphate at 70° C. Gelatinous precipitates obtained was further digested for 1 h at 70° C. After 1 h, the gelatinous precipitate was filtered, washed with water and dried at room temperature. The dried material was converted to acid form by treating material (5 g) with 1 M HNO₃ (50 mL) for 30 min, with occasional shaking. Sample was then separated from acid by decantation and washed with distilled water for removal of adhering acid. Acid treatment was repeated another four times. Finally, the slurry was filtered, washed with water and dried at room temperature. The dried material was grained in a pestle mortar and was characterized with powder X-ray diffraction (PXRD).

### Preparation of ZrP Supported Palladium (Pd/ZrP) Catalyst

Pd/ZrP was prepared by an adsorption method using palladium nitrate (Pd(NO₃)₂) as the palladium precursor. To prepare 5 wt % Pd/ZrP, Pd(NO₃)₂ (corresponding to 150 mg Pd) was dissolved in 200 mL DI water. To clear dark brown solution, 2.85 g ZrP was added and stirred for 2 h at room temperature. The solid was filtered, washed with water and dried at room temperature overnight. The dried catalyst was grained in a pestle mortar before calcining the catalyst in air at 575° C. for 6 h. PXRD was measured for the materials and compared to the literature report (31).^{Error! Bookmark not defined.} The metal loading was measured using ICP-OES.

### Hydrogenolysis of [Ch][Lys]-Poplar Lignin

Hydrogenolysis of [Ch][Lys]-Poplar lignin was performed in a multi-reactor pressure reactor by Parr in duplicates unless otherwise stated. Typically, a magnetic stir bar containing Parr vessel, 0.35 g of lignin was weighed with 0.1 g catalyst and charged with 10 mL of solvent. The vessel was sealed and any residual air was removed by purging with N₂ gas (at least three times). Finally, the reactor was pressurized at 18 bar N₂ gas and stirred at 500 rpm. The temperature was raised to the desired temperature (120° C.-300° C.) and heated for a given time (1-22 h). The reaction time was considered once the desired temperature was achieved. After the passage of given time, the reactor was cooled immediately using an ice bath. The gaseous product was collected (at about 30° C.) in a Tedlar bag and stored for analysis. The reaction mixture was centrifuged to separate solids and liquid. The solids were washed with methanol and dried at 40° C. in air. The liquid thus obtained was concentrated in a vacuum oven at 40° C. The mass of liquid and solid fractions was recorded and used for the yield calculation. To calculate the mass of unreacted solid and char, mass of the catalyst used (considering 95% recovery) was subtracted from the total mass of the solids obtained. The yield of gas was calculated as, [100−(% solid+% liquid)]. The dried liquid layer was used for GC-MS, GPC, and NMR analysis. The dried solids were characterized using PXRD and CHN analysis.

### Reusability of the Catalyst

The solids obtained included both unreacted lignin, char, and the catalyst. To reuse the catalyst, the obtained solids were ground in a pestle mortar and calcined at 575° C. for 6 h. The obtained solids were further grained and characterized using PXRD and ICP to compare with the fresh catalyst.

### GC-MS

For GC-MS analysis, the liquid layer was dissolved in 0.5 mL ethyl acetate and about 18 mg of decane was added to the mixture. The mixture was vortexed well to dissolve the dried liquid layer. The obtained solution was filtered using 0.2 um PTFE filter units before analyzing using Agilent Technologies Inc. 6890N GC equipped with a Agilent mass secretive detector MS 5973N. Helium was used as a carrier gas at a flow rate of 1 mL min⁻¹, while DB-5 ms (30 m×0.25 mm×0.25 um) capillary column was employed for the separation of the degraded lignin products. The oven temperature was initially set at 70° C. and held for 2 min and was increased to 120° C. at a rate of 10° C. min⁻¹ and held for 8 min. Further, the oven temperature was increased to 280° C. at a rate of 10° C. min⁻¹ held for 5 min, followed by an increase to 300° C. at a rate of 7° C. min⁻¹ with a final hold of 3 min. The total run time was 41.86 min. The identification of the lignin degradation products was carried out according to the retention time retrieved from the inbuilt library of the NIST MS Search 2.0 and validated with commercial standards. The relative composition of the identified compounds was determined based on the calibration curve of the commercial standard using decane as an internal standard.

### Statistical Analysis

All the experiments were performed in duplicates. Analysis of variance (ANOVA) was done using JMP 15 (SAS Institute, Inc., Cary, N.C.). To compare the means of oil, solid, and gas yield of poplar samples with respect to temperature a Tukey-Kramer HSD method was implemented at α=0.05.

### Catalyst and Solids Characterization

Rigaku MiniFlex 6G 6th Generation Benchtop X-ray Diffractometer equipped with a 600 W sealed source Cu tube and a HyPix-400MF Hybrid Pixel Array 0D/1/D/2D detector was used for collecting powder X-ray diffraction (PXRD) data. Data collection and analysis was done with SmartLab Studio II. Amounts of palladium loaded on the support was analyzed on a Perkin Elmer Optima 7000 DV Inductively Coupled Plasma-optical emission spectroscopy (ICP-OES) instrument. The elemental (CHN) analysis of the lignin and solid residues after the catalytic run was performed on a Thermo Scientific Flash Smart CHNS analyzer.

### PiMS

The gaseous fraction was collected in 1 L polypropylene Tedlar bags (Restek), transferred to stainless steel cylinders, and diluted (typically by a factor of 4-5) with pure (99.9999%) He. The samples were then analyzed with a home-built photoionization mass spectrometer (49). A broadband H2 discharge lamp was employed as the source of ionizing photons with either a MgF or LiF filter, providing high-energy cutoff wavelengths of about 120 nm (10.2 eV) or 105 nm (11.8 eV), respectively. This results in soft near-threshold photoionization with no significant cation fragmentation, from which small chemical species can be unambiguously assigned based on the parent ion chemical formula.

### HSQC NMR

[Ch][Lys]-Poplar lignin was ground with a mixer mill (Qiagen MM300 Mixer, Retsch) using 2 mm diameter stainless steel balls and 30 s⁻¹ mixing frequency for 15 min. The ground material was dispersed in DMSO-d₆ and allowed to stand overnight to extract lignin. For the lignin oil after hydrogenolysis, the dried oil was directly dissolved in DMSO-d₆ for the NMR analysis. The 2D heteronuclear single quantum coherence (HSQC) spectra were collected on a Bruker Avance I 800 MHz spectrometer equipped with a TXI probe at 310K. A standard Bruker pulse sequence (hsqcetgpsisp2.2) was used with the following parameters which are typical for plant cell wall samples. HSQC spectra were collected from 11 to −1 ppm in F₂ (¹H) dimension with 1024 data points for 53 ms acquisition time, and from 165 to −10 ppm in F (¹³C) dimension with 256 data points for 3.5 ms acquisition time. A total of 256 scans were recorded for each t1 point with a pulse delay of 1 s. The central DMSO solvent peak was used as a reference for the chemical shift calibration for all samples (δ_{C} 39.5 ppm, δ_{H} 2.5 ppm). All HSQC spectra were processed using typical 90° sine square apodization in both F₂ and F₁ dimensions and the contours were integrated in the MestreNOVA software (v.14). Peaks were assigned according to published data.

### Gel Permeation Chromatography

GPC was used to determine the relative MW distribution of lignin in substrate as described previously (50). Typically, 10 mg of [Ch][Lys]-Poplar lignin was incubated in 2.5 mL of 92:8 (v/v) acetic acid and acetyl bromide mixture and stirred at 50° C. for 2 h to dissolve lignin. (CAUTION: Acetyl bromide must be operated in fume hood with appropriate PPE. See MSDS for further instructions) Excess of acetyl bromide and acetic acid were removed with a rotary evaporator connected to a high vacuum pump and a cold trap. This acetylated lignin was immediately dissolved in tetrahydrofuran (THF) and filtered through 0.2 μm polytetrafluoroethylene (PTFE) filters (GE Healthcare Life Sciences, USA). GPC analysis was conducted using a Tosoh Ecosec HLC-8320GPC (Griesheim, Germany) equipped with Agilent PLgel 5 μm Mixed-D and a Diode array detector. THE (spiked with 250 ppm of butylated hydroxytoluene) was used as the eluent (1 mL min⁻¹, column temperature 40° C.). The GPC standards, which contained polystyrene ranging from 162 to 69,650 g mol⁻¹, were purchased from Agilent and used for calibration. The oil after hydrogenolysis was directly dissolved in THE (completely soluble) and after filtration through 0.2 μm PTFE filter units were analyzed on the same system as described above.

TABLE 2
Retention times of various molecules as identified using
commercial standards.

| Chemical Name | Chemical Structure | |
|---|---|---|
| 3-methylcyclopent- 2-en-1-one | 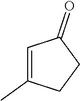 | 5.092 |
| Phenol | 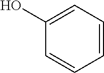 | 6.055 |
| Decane | 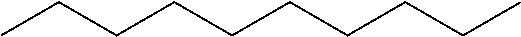 | 6.435 |
| Dimethylsuccinate | 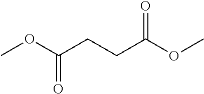 | 6.739 |
| 4-methylphenol | 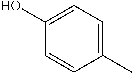 | 7.466 |
| guaiacol | 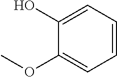 | 7.713 |
| 4-ethylanisole | 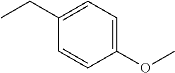 | 8.186 |
| 4-ethylphenol | 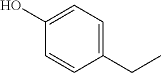 | 9.245 |
| 4-methylguaiacol | 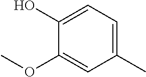 | 9.811 |
| 4-ethylguaiacol | 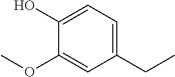 | 12.475 |
| 2,6-dimethoxyphenol | 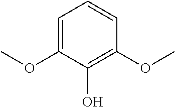 | 15.652 |
| 4-propylguaiacol | 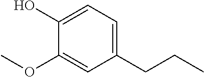 | 16.314 |
| 3,4,5- trimethoxy- benzaldehyde | 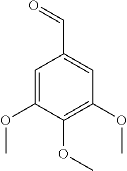 | 20.150 |
| Pyrogallol | 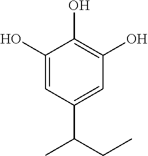 | 20.779 |

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for depolymerizing a lignin, said method comprising: (a) providing a metal catalyst, and (b) contacting a lignin to the metal catalyst, such that the metal catalyst depolymerizes at least a portion of the lignin into one or more lignin monomers.

2. The method of claim 1, wherein the metal catalyst is a metal-nanoparticle (NP) catalyst.

3. The method of claim 1, wherein the metal catalyst is supported on a solid support.

4. The method of claim 2, wherein the NP is a multimetallic NP, such as a bimetallic NP or trimetallic NP.

5. The method of claim 1, wherein the metal catalyst is Ni, Cu, Cr, Co, Fe, Ru, Rh, Pd, Pt, Au, Re, and/or Ir, or a mixture thereof.

6. The method of claim 5, wherein the metal catalyst is palladium.

7. The method of claim 1, wherein the metal catalyst is supported on or bound to a support.

8. The method of claim 7, wherein the support is an acid support or a basic support.

9. The method of claim 7, wherein the support comprises zirconium phosphate.

10. The method of claim 7, wherein the metal catalyst is solid acid support comprising zirconium phosphate.

11. The method of claim 1, wherein the one or lignin monomers are a phenol or guaiacol, or a derivative thereof.

12. The method of claim 1, wherein the one or more lignin monomers comprise a

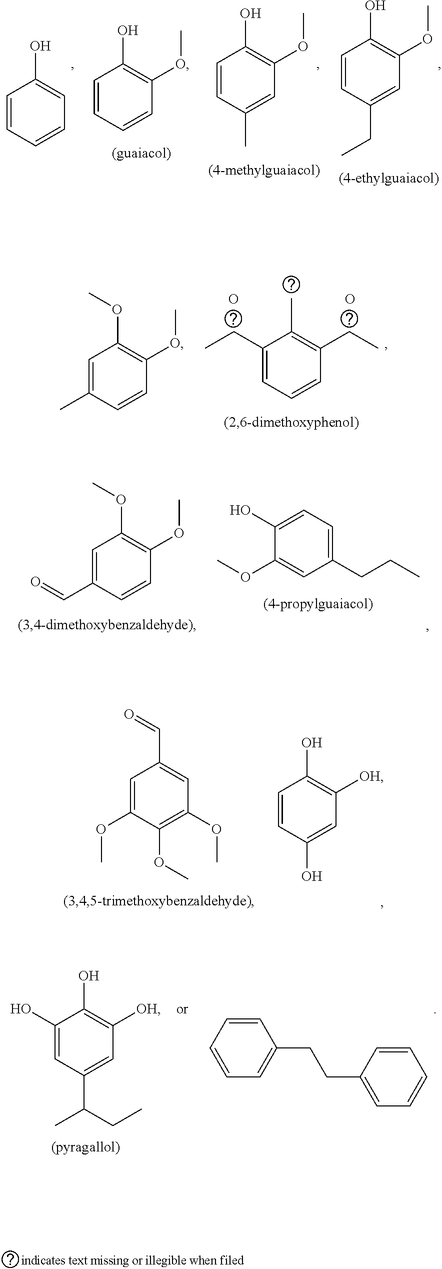

13. The method of claim 12, wherein the one or more lignin monomers comprise [chemical structure]
